SEQUENCE LISTING (52 sequences)

SEQ ID NO: 1 - protein, 145 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSQDMLWQLDLSPGC

SEQ ID NO: 2 - protein, 145 aa, Artificial Sequence
  Human Leptin Analog
VNITKVQDDTKTLIKTIVTRINDISHTQSVSSKQKVTGLDFIPGLHNITTLSKMDQTLAVYQQILTSMNSTNVIQISNDLENLRDLLHVLAFSKSCHLPWASGLETLDSLGGVLEASGYSTEWALSRLQGSLQDMLWQLDLSPGC

SEQ ID NO: 3 - protein, 145 aa, Artificial Sequence
  Human Leptin Analog 2
VPIQKVQDDTKTLIKTIVTRINDISHTQSVSSKQKVTGLDFIPGLHNITTLSKMDQTLAVYQQILTSMNSTNVIQISNDLENLRDLLHVLANSTSCHLPWANGTETLDSLGGVLEASGYSTEWALSRLQGSLQDMLWQLDLSPGC

SEQ ID NO: 4 - protein, 146 aa, Canine
VPIRKVQDDTKTLIKTIVARINDISHTQSVSSKQRVAGLDFIPGLQPVLSLSRMDQTLAIYQQILNSLHSRNVVQISNDLENLRDLLHLLASSKSCPLPRARGLETFESLGGVLEASLYSTEVVALNRLQAALQDMLRRLDLSPGC

SEQ ID NO: 5 - protein, 146 aa, Artificial Sequence
  Canine Leptin Analog
VPIRKVQDDTKTLIKTIVARINDISHTQSVSSKQRVAGLDFIPGLQNVTSLSRMDQTLAIYQQILNSLNSTNVVQISNDLENLRDLLHLLASSKSCPLPRANGTETFESLGGVLEASLYSTEVVALNRLQAALQDMLRRLDLSPGC

SEQ ID NO: 6 - protein, 146 aa, Feline
VPIRKVQDDTKTLIKTIVTRINDISHTQSVSSKQRVAGLDFIPGLHPVLSLSKMDQTLAIYQQILTGLPSRNVVQISNDLENLRDLLHLLASSKNCPLPRARGLETLESLGGVLEASLYSTEVVALSRLQASLQDMLWRLDLSPGC

SEQ ID NO: 7 - protein, 146 aa, Feline
VPIRKVQDDTKTLIKTIVTRINDISHTQSVSSKQRVAGLDFIPGLHNVTSLSKMDQTLAIYQQILTGLNSTNVVQISNDLENLRDLLHLLASSKNCPLPRANGTETLESLGGVLEASLYSTEVVALSRLQASLQDMLWRLDLSPGC

SEQ ID NO: 8 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 232 aa, Artificial Sequence
  Modified Human IgG2 Fc
DKTVERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 10 - protein, 229 aa, Artificial Sequence
  Modified Human IgG4 Fc
AESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 11 - protein, 237 aa, Artificial Sequence
  Modified Canine IgGA Fc
DKPVFNESRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 12 - protein, 229 aa, Canine
PRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 13 - protein, 233 aa, Artificial Sequence
  Modified Canine IgGD Fc
PKESTSKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 14 - protein, 232 aa, Artificial Sequence
  Modified Canine IgGD Fc-2
PKESTSKCPPCPVPEAAGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 15 - protein, 230 aa, Feline
GPKPSDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 16 - protein, 237 aa, Feline
PKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 17 - protein, 19 aa, Artificial Sequence
  Angiopep-2 Seq 97
TFFYGGSRGKRNNFKTEEY

SEQ ID NO: 18 - protein, 19 aa, Artificial Sequence
  Angiopep-2 Seq 117
YEETKFNNRKGRSGGYFFT

SEQ ID NO: 19 - protein, 264 aa, Homo sapiens
ASEVQLLESGGDLIQSGGSLRLSCAASGFTVTSNYMNWVRQAPGKGLEWVSLIYSGGSTSYADSVKGRFTISRDNSNNTLYLQMNSLRAEDTAVYYCARGRPNWYFDLWGRGTLVTVSSGILGSGGGGSGGGGSGGGGSQSVLTQPASVSGSPGQSITISCTGTSRDIGAYNYVSWYQQHPGKAPKVMIYDVSKRPSGVPDRFSGSKSGNTASLTISGLQAEDEADYYCSSYTSSSTPHVVFGGGTKLTVLSGILEQKLISEED

SEQ ID NO: 20 - protein, 250 aa, Canine
EVQLVESGGDLVKPGGSLRLYCVASGFTVTSNYMNWVRQAPGKGLQWVALIYSGGSTSYADSVKGRFTISRDNARNTLYLQMDSLRSEDTAVYYCARGRPNWYFDLWGQGTLVTVSSASTGGGGSGGGGSGGGGSGSQSVLTQPASVSGSLGQRVTISCTGTSRDIGAYNYVSWYQQLPGRSPRTLIYDVSKRPSGVPDRFSGSRSGNTATLTISGLQAEDEADYYCSSYTSSSTPHVVFGGGTHLTVLG

SEQ ID NO: 21 - protein, 218 aa, Canine
QSVLTQPASVSGSLGQRVTISCTGTSRDIGAYNYVSWYQQLPGRSPRTLIYDVSKRPSGVPDRFSGSRSGNTATLTISGLQAEDEADYYCSSYTSSSTPHVVFGGGTHLTVLGQPKASPSVTLFPPSSEELGANKATLVCLISDFYPSGVTVAWKASGSPVTQGVETTKPSKQSNNKYAASSYLSLTPDKWKSHSSFSCLVTHEGSTVEKKVAPAECS

SEQ ID NO: 22 - protein, 222 aa, Canine
EVQLVESGGDLVKPGGSLRLYCVASGFTVTSNYMNWVRQAPGKGLQWVALIYSGGSTSYADSVKGRFTISRDNARNTLYLQMDSLRSEDTAVYYCARGRPNWYFDLWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCK

SEQ ID NO: 23 - protein, 228 aa, Canine
EVQLVESGGDLVKPGGSLRLYCVASGFTVTSNYMNWVRQAPGKGLQWVALIYSGGSTSYADSVKGRFTISRDNARNTLYLQMDSLRSEDTAVYYCARGRPNWYFDLWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCP

SEQ ID NO: 24 - protein, 448 aa, Canine
EVQLVESGGDLVKPGGSLRLYCVASGFTVTSNYMNWVRQAPGKGLQWVALIYSGGSTSYADSVKGRFTISRDNARNTLYLQMDSLRSEDTAVYYCARGRPNWYFDLWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 25 - protein, 608 aa, Canine
MKWVTFISLFFLFSSAYSRGLVRREAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYARRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCAEDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 26 - protein, 608 aa, Feline; identical to SEQ ID NO: 25

SEQ ID NO: 27 - protein, 609 aa, Homo sapiens [sequence omitted]

SEQ ID NO: 28 - protein, 25 aa, Artificial Sequence
  Human GLP-1 Analog
HGEGTFTSDVSSYLEEQAAKEFIAW

SEQ ID NO: 29 - protein, 39 aa, Homo sapiens
HGEGTFTSDLSKQMEEEAVRLFIEWLKNGGPSSGAPPPS

SEQ ID NO: 30 - protein, 29 aa, Homo sapiens
HSQGTFTSDYSKYLDSRRAQDFVQWLMNT

SEQ ID NO: 31 - protein, 42 aa, Homo sapiens
YAEGTFISDYSIAMDKIHQQDFVNWLLAQKGKKNDWKHNITQ

SEQ ID NO: 32 - protein, 37 aa, Homo sapiens
HSQGTFTSDYSKYLDSRRAQDFVQWLMNTKRNRNNIA

SEQ ID NO: 33 - protein, 29 aa, Artificial Sequence
  Human Oxyntomodulin Analog-1
  MISC_FEATURE (2)..(2): Xaa = aminoisobutyric acid
HXQGTFTSDYSKYLDEKRAKEFVQWLMNT

SEQ ID NO: 34 - protein, 29 aa, Artificial Sequence
  Human Oxyntomodulin Analog-2
  MISC_FEATURE (2)..(2): Xaa = aminoisobutyric acid
HXQGTFTSDYSKYLDEKRAKEFVQWLMNT

SEQ ID NO: 35 - protein, 29 aa, Artificial Sequence
  Human Oxyntomodulin Analog-3
  MISC_FEATURE (2)..(2): Xaa = isobutyric acid
HXQGTFTSDYSKYLDEQAAKEFICWLMNT

SEQ ID NO: 36 - protein, 5 aa, Artificial Sequence
  Peptide Linker
GGGGS

SEQ ID NO: 37 - protein, 10 aa, Artificial Sequence
  Peptide Linker 2
GGGGSGGGGS

SEQ ID NO: 38 - protein, 15 aa, Artificial Sequence
  Peptide Linker 3
GGGGSGGGGSGGGGS

SEQ ID NO: 39 - protein, 20 aa, Artificial Sequence
  Peptide Linker 4
GGGGSGGGGSGGGGSGGGGS

SEQ ID NO: 40 - protein, 12 aa, Artificial Sequence
  Peptide Linker 5
AEAAAKEAAAKA

SEQ ID NO: 41 - protein, 383 aa, Artificial Sequence
  Fc-Leptin Fusion Protein-1
PKESTSKCPPCPVPEAAGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGKGGGGSVPIRKVQDDTKTLIKTIVARINDISHTQSVSSKQRVAGLDFIPGLQPVLSLSRMDQTLAIYQQILNSLHSRNVVQISNDLENLRDLLHLLASSKSCPLPRARGLETFESLGGVLEASLYSTEVVALNRLQAALQDMLRRLDLSPGC

SEQ ID NO: 42 - protein, 383 aa, Artificial Sequence
  Fc-Leptin Fusion Protein-2
PKESTSKCPPCPVPEAAGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGKGGGGSVPIRKVQDDTKTLIKTIVARINDISHTQSVSSKQRVAGLDFIPGLQNVTSLSRMDQTLAIYQQILNSLNSTNVVQISNDLENLRDLLHLLASSKSCPLPRANGTETFESLGGVLEASLYSTEVVALNRLQAALQDMLRRLDLSPGC

SEQ ID NO: 43 - protein, 401 aa, Artificial Sequence
  ScFv-Leptin Fusion Protein
EVQLVESGGDLVKPGGSLRLYCVASGFTVTSNYMNWVRQAPGKGLQWVALIYSGGSTSYADSVKGRFTISRDNARNTLYLQMDSLRSEDTAVYYCARGRPNWYFDLWGQGTLVTVSSASTGGGGSGGGGSGGGGSGSQSVLTQPASVSGSLGQRVTISCTGTSRDIGAYNYVSWYQQLPGRSPRTLIYDVSKRPSGVPDRFSGSRSGNTATLTISGLQAEDEADYYCSSYTSSSTPHVVFGGGTHLTVLGGGGGSVPIRKVQDDTKTLIKTIVARINDISHTQSVSSKQRVAGLDFIPGLQNVTSLSRMDQTLAIYQQILNSLNSTNVVQISNDLENLRDLLHLLASSKSCPLPRANGTETFESLGGVLEASLYSTEVVALNRLQAALQDMLRRLDLSPGC

SEQ ID NO: 44 - protein, 373 aa, Artificial Sequence
  Fab-Leptin Fusion Protein, HC
EVQLVESGGDLVKPGGSLRLYCVASGFTVTSNYMNWVRQAPGKGLQWVALIYSGGSTSYADSVKGRFTISRDNARNTLYLQMDSLRSEDTAVYYCARGRPNWYFDLWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKGGGGSVPIRKVQDDTKTLIKTIVARINDISHTQSVSSKQRVAGLDFIPGLQNVTSLSRMDQTLAIYQQILNSLNSTNVVQISNDLENLRDLLHLLASSKSCPLPRANGTETFESLGGVLEASLYSTEVVALNRLQAALQDMLRRLDLSPGC

SEQ ID NO: 45 - protein, 369 aa, Artificial Sequence
  Fab-Leptin Fusion Protein, LC
QSVLTQPASVSGSLGQRVTISCTGTSRDIGAYNYVSWYQQLPGRSPRTLIYDVSKRPSGVPDRFSGSRSGNTATLTISGLQAEDEADYYCSSYTSSSTPHVVFGGGTHLTVLGQPKASPSVTLFPPSSEELGANKATLVCLISDFYPSGVTVAWKASGSPVTQGVETTKPSKQSNNKYAASSYLSLTPDKWKSHSSFSCLVTHEGSTVEKKVAPAECSGGGGSVPIRKVQDDTKTLIKTIVARINDISHTQSVSSKQRVAGLDFIPGLQNVTSLSRMDQTLAIYQQILNSLNSTNVVQISNDLENLRDLLHLLASSKSCPLPRANGTETFESLGGVLEASLYSTEVVALNRLQAALQDMLRRLDLSPGC

SEQ ID NO: 46 - protein, 599 aa, Artificial Sequence
  Antibody-Leptin Fusion Protein, HC
EVQLVESGGDLVKPGGSLRLYCVASGFTVTSNYMNWVRQAPGKGLQWVALIYSGGSTSYADSVKGRFTISRDNARNTLYLQMDSLRSEDTAVYYCARGRPNWYFDLWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGKGGGGSVPIRKVQDDTKTLIKTIVARINDISHTQSVSSKQRVAGLDFIPGLQNVTSLSRMDQTLAIYQQILNSLNSTNVVQISNDLENLRDLLHLLASSKSCPLPRANGTETFESLGGVLEASLYSTEVVALNRLQAALQDMLRRLDLSPGC

SEQ ID NO: 47 - protein, 394 aa, Artificial Sequence
  Fc-Leptin Fusion Protein A
MGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGKGGGGSGGGGSGGGGSVPIRKVQDDTKTLIKTIVARINDISHTQSVSSKQRVAGLDFIPGLQPVLSLSRMDQTLAIYQQILNSLHSRNVVQISNDLENLRDLLHLLASSKSCPLPRARGLETFESLGGVLEASLYSTEVVALNRLQAALQDMLRRLDLSPGC

SEQ ID NO: 48 - protein, 394 aa, Artificial Sequence
  Fc-Leptin Fusion Protein B
MGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGKGGGGSGGGGSGGGGSVPIRKVQDDTKTLIKTIVARINDISHTQSVSSKQRVAGLDFIPGLQPVLSLSRMDQTLAIYQQILNSLHSRNVVQISNDLENLRDLLHLLASSKSCPLPRARGLETFESLGGVLEASLYSTEVVALNRLQAALQDMLRRLDLSPGC

SEQ ID NO: 49 - protein, 985 aa, Artificial Sequence
  Albumin-Fc-Leptin Fusion Protein C
MEAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYARRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCAEDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALVGGGGSGGGGSGGGGSCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGKGGGGSGGGGSGGGGSVPIRKVQDDTKTLIKTIVARINDISHTQSVSSKQRVAGLDFIPGLQPVLSLSRMDQTLAIYQQILNSLHSRNVVQISNDLENLRDLLHLLASSKSCPLPRARGLETFESLGGVLEASLYSTEVVALNRLQAALQDMLRRLDLSPGC

SEQ ID NO: 50 - DNA, 1188 bp, Artificial Sequence
  Fc-Leptin Fusion Protein A Coding Sequence
atgggtcgtgttcctcgtccgccagattgtccaaaatgtcctgctcctgaaatgctgggtggcccgtcggtctttatctttccgccgaagccgaaagataccctgctgatcgcgcgtacgccggaagtgacgtgcgttgtcgttgacctggacccggaagatccagaggtccagatttcctggtttgtcgacggtaagcaaatgcagaccgccaaaacccagccgcgcgaggaacagttcaacggtacctaccgtgtcgtgagcgtcctcccgattggtcaccaagactggctgaaaggtaagcaattcacctgtaaggttaacaataaagcactgccgagcccgatcgaacgtacgattagcaaggctcgcggccaggctcatcaaccgtccgtgtatgttctgccgccgagccgcgaggaactgagcaagaatacggtcagcttgacctgtctgattaaagacttttttccgccggatatcgacgttgaatggcagtcgaacggccaacaagagccggaaagcaaataccgtaccactccgccgcagctggatgaagatggcagctattttctgtactctaaactgagcgtggacaaaagccgctggcaacgtggtgatacgttcatttgcgccgttatgcacgaggcactgcacaatcactacacccaggaaagcctgagccacagcccgggcaagggtggtggcggcagcggtggtggcggctcgggtggcggtggtagcgtgccgattcgtaaggtccaagatgataccaagacgctgattaaaaccatcgtcgcgcgcatcaacgacatcagccatactcagagcgtgagctccaaacagcgtgtggcgggtctggactttatcccgggtctgcagccggttctgtccctgagccgtatggatcaaaccctggcaatttatcagcaaattttgaattctctgcacagccgtaacgttgtccaaatttccaatgacctggagaatctgcgcgacttgctgcatttgctggcgagctctaaaagctgcccgctgccgcgtgcccgcggcttggagactttcgagtccctgggtggcgtgctggaagcatctctgtacagcaccgaagttgtggcattaaaccgtctgcaagcggcgctgcaagacatgctgcgtcgcctggacttgagcccgggttgctgataa

SEQ ID NO: 51 - DNA, 1188 bp, Artificial Sequence
  Fc-Leptin Fusion Protein B Coding Sequence
atgggtcgtgttcctcgtccgccagattgtccaaaatgtcctgctcctgaaatgctgggtggcccgtcggtctttatctttccgccgaagccgaaagataccctgtatatcacgcgtgagccggaagtgacgtgcgttgtcgttgacctggacccggaagatccagaggtccagatttcctggtttgtcgacggtaagcaaatgcagaccgccaaaacccagccgcgcgaggaacagttcaacggtacctaccgtgtcgtgagcgtcctcccgattggtcaccaagactggctgaaaggtaagcaattcacctgtaaggttaacaataaagcactgccgagcccgatcgaacgtacgattagcaaggctcgcggccaggctcatcaaccgtccgtgtatgttctgccgccgagccgcgaggaactgagcaagaatacggtcagcttgacctgtctgattaaagacttttttccgccggatatcgacgttgaatggcagtcgaacggccaacaagagccggaaagcaaataccgtaccactccgccgcagctggatgaagatggcagctattttctgtactctaaactgagcgtggacaaaagccgctggcaacgtggtgatacgttcatttgcgccgttatgcacgaggcactgcacaatcactacacccaggaaagcctgagccacagcccgggcaagggtggtggcggcagcggtggtggcggctcgggtggcggtggtagcgtgccgattcgtaaggtccaagatgataccaagacgctgattaaaaccatcgtcgcgcgcatcaacgacatcagccatactcagagcgtgagctccaaacagcgtgtggcgggtctggactttatcccgggtctgcagccggttctgtccctgagccgtatggatcaaaccctggcaatttatcagcaaattttgaattctctgcacagccgtaacgttgtccaaatttccaatgacctggagaatctgcgcgacttgctgcatttgctggcgagctctaaaagctgcccgctgccgcgtgcccgcggcttggagactttcgagtccctgggtggcgtgctggaagcatctctgtacagcaccgaagttgtggcattaaaccgtctgcaagcggcgctgcaagacatgctgcgtcgcctggacttgagcccgggttgctgataa

SEQ ID NO: 52 - DNA, 2955 bp, Artificial Sequence
  Albumin-Fc-Leptin Fusion Protein C Coding Sequence
atggaagcctacaagtcagaaatcgcccaccgttataatgatctgggcgaggagcattttcgtggtctggtgctggttgcgttcagccaatacctgcaacagtgcccgtttgaagatcacgtcaagctggcgaaagaggtgacggagtttgcgaaggcgtgcgcagccgaagaaagcggcgcgaactgtgacaaatccttgcacaccctgttcggtgacaaattgtgtaccgtggcaagcttacgcgacaaatacggtgacatggcggactgctgcgaaaaacaagaaccggaccgcaatgaatgttttctggctcacaaagacgataatccgggcttcccgccgcttgttgctccggaaccggacgctctgtgcgcggcgttccaggataacgaacaactgtttctgggcaagtacctgtacgaaattgctcgtcgccatccgtacttttacgcgccagagttgctgtattacgcgcaacaatataaaggtgttttcgcggagtgctgccaagcggcggataaggcggcctgcctcggtccgaagattgaggcactgcgcgaaaaagttctgttgagcagcgccaaagagcgcttcaaatgcgcgagcttgcagaaattcggcgaccgcgcgttcaaagcatggagcgtcgcgcgtctgagccaacgttttcctaaggcagactttgcggagattagcaaggtcgtgacggacctgaccaaggtccacaaggaatgctgtcacggtgacttattggagtgtgcggatgaccgtgcggacctggcaaaatacatgtgtgagaatcaagattccatcagcaccaagctgaaagagtgctgtgataagccggttctggaaaaaagccaatgcttggcggaggtggagcgtgatgagctgccaggtgatctgccgagcctggctgccgactttgttgaggataaggaagtttgtaagaattatcaagaggcgaaagacgttttcctgggcacgtttttgtatgagtacgcgcgtcgccacccggaatacagcgtcagcctgttgctgcgcctggcaaaggagtacgaggctaccctggaaaaatgctgcgccaccgatgacccgccgacctgttacgcaaaggtgctggacgaattcaagccgctggtcgatgagccgcaaaatctggtgaaaaccaactgcgagttgttcgagaaactgggcgagtacggcttccaaaacgcgctgctggtccgttacacgaagaaggcgccgcaagtctcgaccccgacgctggtcgaggtcagccgtaaacttggtaaggtgggtaccaaatgctgtaaaaaaccggaatctgagcgtatgagctgcgcggaggactttctgagcgttgtgctgaaccgtctgtgcgttctgcacgaaaaaacccctgttagcgaacgcgttaccaaatgctgcagcgaaagcctggttaatagacgtccgtgcttttccggtctggaagtcgacgagacgtacgtgccgaaggagttcaatgcggaaacctttacctttcatgcagacttgtgcacgctgccggaggcggagaagcaggtgaaaaagcaaaccgcactggtggaattactgaagcacaagccaaaagcgaccgatgaacaattaaagaccgttatgggtgattttggtgcgtttgtggagaaatgttgtgcggctgagaataaggaaggctgctttagcgaggagggtccgaagctggtcgcagcagcgcaggcagctttggttggtggtggtggcagcggcggtggtggcagcggcggtggtggcagctgtccaaaatgtcctgctcctgaaatgctgggtggcccgtcggtctttatctttccgccgaagccgaaagataccctgtacatcacgcgtgagccggaagtgacgtgcgttgtcgttgacctggacccggaagatccagaggtccagatttcctggtttgtcgacggtaagcaaatgcagaccgccaaaacccagccgcgcgaggaacagttcaacggtacctaccgtgtcgtgagcgtcctcccgattggtcaccaagactggctgaaaggtaagcaattcacctgtaaggttaacaataaagcactgccgagcccgatcgaacgtacgattagcaaggctcgcggccaggctcatcaaccgtccgtgtatgttctgccgccgagccgcgaggaactgagcaagaatacggtcagcttgacctgtctgattaaagacttttttccgccggatatcgacgttgaatggcagtcgaacggccaacaagagccggaaagcaaataccgtaccactccgccgcagctggatgaagatggcagctattttctgtactctaaactgagcgtggacaaaagccgctggcaacgtggtgatacgttcatttgcgccgttatgcacgaggcactgcacaatcactacacccaggaaagcctgagccacagcccgggcaagggtggtggcggcagcggtggtggcggctcgggtggcggtggtagcgtgccgattcgtaaggtccaagatgataccaagacgctgattaaaaccatcgtcgcgcgcatcaacgacatcagccatactcagagcgtgagctccaaacagcgtgtggcgggtctggactttatcccgggtctgcagccggttctgtccctgagccgtatggatcaaaccctggcaatttatcagcaaattttgaattctctgcacagccgtaacgttgtccaaatttccaatgacctggagaatctgcgcgacttgctgcatttgctggcgagctctaaaagctgcccgctgccgcgtgcccgcggcttggagactttcgagtccctgggtggcgtgctggaagcatctctgtacagcaccgaagttgtggcattaaaccgtctgcaagcggcgctgcaagacatgctgcgtcgcctggacttgagcccgggttgc

(19) United States
(12) Patent Application Publication
(10) Pub. No.: US 2018/0009869 A1
(43) Pub. Date: Jan. 11, 2018

(54) Fusion Protein Comprising Leptin and Methods for Producing and Using the Same

(71) Applicant: AskGene Pharma, Inc., Camarillo, CA (US)

(72) Inventors: Yuefeng Lu, Newbury Park, CA (US); Jianfeng Lu, Oak Park, CA (US); Lan Yang, Camarillo, CA (US); Lu Li, Camarillo, CA (US); Kurt SHANEBECK, Camarillo, CA (US); Lei Liu, Thousand Oaks, CA (US)

(73) Assignee: AskGene Pharma, Inc., Camarillo, CA (US)

(21) Appl. No.: 15/644,764

(22) Filed: Jul. 8, 2017

Related U.S. Application Data
(60) Provisional application No. 62/360,271, filed on Jul. 8, 2016.

(51) Int. Cl. C07K 14/575; C07K 7/08; C07K 14/76; A61K 38/00

(52) U.S. Cl. CPC C07K 14/5759; C07K 14/76; C07K 7/08; A61K 38/00; C07K 2319/30; C07K 2319/31

(57) ABSTRACT

The present invention provides fusion proteins comprising leptin and a second protein. The presence of the second protein provides increased biological activity and/or increased half-life in vivo. The present invention also provides human, canine and feline leptin molecules fused to peptides, antibodies or antibody fragments which enhances the abilities of the leptin molecules to transport through the blood-brain-barrier (BBB). The present invention also provides fusion proteins further comprising a peptide agonist that is capable of binding to and stimulate one, two or all three of the following receptors: GLP-1 receptor, Glucagon receptor, and GIP receptor. Also disclosed is a method of production such fusion proteins through recombinant technologies. The invention further discloses a pharmaceutical composition comprising one of the fusion proteins as an active intergradient as well as a method for using such a pharmaceutical composition to treat diseases in dogs, cats and humans.

### CROSS-REFERENCE TO RELATED APPLICATIONS

This application claims the priority benefit of U.S. Provisional Application No. 62/360,271, filed Jul. 8, 2016, which is incorporated herein by reference in its entirety.

### FIELD OF THE INVENTION

The present invention relates to a fusion (i.e., a chimeric) protein that comprises an antibody or a fragment thereof and a leptin. The present invention also relates to a pharmaceutical composition comprising the same and a method for producing and using the same. In particular, the present invention relates to a preparation and application of FC region and the immunoglobulin-containing leptin (“leptin”) fusion protein.

### BACKGROUND OF THE INVENTION

Metabolic disorders in pets are associated with some of the major physiological disorders such as diabetes, hypertension, heart disease and certain types of cancer as well as other metabolic disorder related diseases. For example, in the United States alone, it is estimated that 52.7%, or about 43.8 million of dogs are overweight or obese, among them an estimated 17.6% or about 13.9 million dogs are estimated to be obese. In addition, an estimated 57.9%, or about 55 million of cats in the U.S. are overweight or obese of which 28.1% or about 26.2 million cats are estimated to be obese. There are more and more signs that obesity has become a serious health problem for household companion animals in the U.S. and worldwide.

Obesity is a multifactorial phenotype that can be a result of various factors, e.g., physiological, psychological, genetic and environmental. One particular factor associated with obesity is the obese (ob) gene, which has been cloned.

In normal mice, the ob gene encodes a hormone called leptin. Leptin binds to the receptor messenger exercise function, a longer form of the receptor having a cytoplasmic domain capable of signal transduction. Through receptor-hormone mechanism, it is believed that adipose tissue informs the brain about the status of energy storage by releasing leptin in to the blood stream where leptin crosses the blood-brain barrier to the leptin receptor in the hypothalamus. When the brain receives energy storage information, it commands the body by reducing food intake and/or increasing energy consumption to be adjusted accordingly.

Morbid mice (i.e., ob/ob homozygote) are obese mouse having two mutant ob alleles homozygous. The mutant alleles produce truncated leptin, which is non-functional and may degrade quickly in the body. Mice with two mutant ob alleles (ob/ob mice) lack functional leptin and results in lethargy, hypothermia, high blood sugar, high blood insulin and infertility. In humans, although the majority of obese patients have been reported to have high levels of circulating leptin, there is a lack of evidence of leptin and show considerably weight gain and obesity-related disorders.

It is believed that administration of recombinant leptin may relieve ob/ob mice lacking leptin various symptoms related to metabolic disorders. For example, it has been shown that daily intraperitoneal injection of leptin reduces food intake, body weight, body fat percentage and serum glucose and insulin concentrations in ob/ob mice. Furthermore, administration of leptin has shown to increase metabolic rate, body temperature and locomotor activity, all of which require energy expenditure. The study also showed treatment with leptin resulted in the reduction of weight, food intake and body fat. Normal mice also benefited from leptin treatment. Others have shown that recombinant leptin can also be used to correct infertility in female and male ob/ob mice.

It is estimated that about 5-10% of obese humans are sensitive to leptin treatment. Unfortunately, current application in the form of multiple daily injections of leptin requires high doses of leptin to achieve the desired clinical result. For example, in recent clinical trials, some volunteers required large doses of leptin injections three times a day for up to six months to see effective results. Without being bound by any theory, it is believed this large dosage requirement and a prolong treatment is required due to a relative ineffectiveness of a low dosage of leptin and/or a relative short serum half-life of leptin. Thus, it is believed that use of natural leptin will require frequent administration of large doses.

This finding is also consistent with the observation in ob/ob mice experiments, which required intraperitoneal injections of 5-20 mg/kg/day of leptin over an extended period to see a significant reduction of body weight in ob/ob mice. One method of achieving a desired physiological plasma level of leptin to overcome this short comings of leptin (i.e., low effectiveness and/or short half-life) in ob/ob mice required continuous subcutaneous infusion of 400 ng/hr leptin.

Again without being bound by any theory, inherent characteristics of leptin, such as its size and its preparation method, appears to be one of the reasons for shortcoming of using leptin for treatment of obesity. For example, a molecular weight of leptin is about 16 kD, which is small enough to be sufficiently removed by kidney filtration. To compensate for a relatively short serum half-life of leptin in the body requires administering a relatively large doses.

In addition, production of smaller proteins, such as leptin, using bacteria can be difficult and problematic. In some cases production of small proteins such as leptin using E. coli can result in proteins being produced as insoluble inclusion bodies. To obtain proteins from inclusion bodies often requires using denaturing agents, e.g., guanidine hydrochloride, to dissolve the inclusion bodies which can result in destruction of some of the desired proteins. In addition, purification steps may also require denaturing conditions. Thus, to produce small functional proteins using bacteria often requires “folding” the proteins under appropriate conditions after isolation. In addition, some proteins including leptin contain intramolecular disulfide bond that must also be reformed or remade. Thus, production of small soluble bioactive molecules using bacteria sometimes requires recovering the produced protein, folding the protein and/or forming intramolecular disulfide bonds.

As a result of such a complicated production process, production of small functional proteins such as leptin using prokaryotes is often difficult, if not impossible. Attempts have been made to improve production of leptin by improving its solubility. For example, one method involves mutating (e.g., replacing) certain amino acid residues with aspartic acid or glutamic acid to improve the leptin isoelectric point (pI) from 5.84 to 5.5 or less. See, for example, U.S. Pat. No. 5,719,266. However, the resulting leptin “derivative” may also be immunogenic in the intended recipient.

Due at least in part to the large dose requirement, inefficiencies in production, short serum half-life and extremely complex process in the production and purification of leptin, there is a need for a method for increasing the yield of leptin. There is also a need for leptin having improved pharmacological properties. While a longer half-life canine leptin analog, in the form of pegylated protein, is known (see PCT publication number WO 2014/165189), there is a continuing need for active and longer half-life versions of canine leptin analogs to treat obesity and related metabolic disorders in dogs.

### SUMMARY OF THE INVENTION

Some aspects of the present invention provide a fusion protein and chimeric protein comprising a leptin that has improved (i) pharmacological properties, (ii) in vivo half-life or (iii) both. In some embodiments, the fusion protein (i.e., the chimeric protein) comprising a leptin has improved half-life.

One particular aspect of the invention provides a fusion protein comprising a first protein that is linked to a second protein. The first protein can be linked to the second protein via a linker or they can be linked directly to one another. Exemplary linkers are include, but are not limited to, SEQ ID NOS:36-40. It should be appreciated that any suitable linkers can be used including polyethylene glycols (PEG) as well as other amino acid chains known to one skilled in the art. The first protein comprises (a) a canine immunoglobulin Fc (“Ig Fc”) region; (b) a canine albumin having amino acid sequence of at least 75% sequence identity to SEQ ID NO:25; (c) a feline Ig Fc region; or (d) a feline albumin having amino acid sequence of at least 75% sequence identity to SEQ ID NO:26. Depending on the identity of the first protein, the second protein can be either a canine leptin protein or a feline leptin protein.

In another aspect of the invention, the first protein comprises a human leptin or its analog with an amino acid sequences with at least 85% sequence identity to SEQ ID NO:1, SEQ ID NO:2 or SEQ ID NO:3, and the second protein comprises a Fc fragment selected from the group consisting of human IgG1 Fc (e.g., SEQ ID NO:8), human IgG2 Fc (e.g., SEQ ID NO:9) and human IgG4 Fc (e.g., SEQ ID NO:10).

Yet another aspect of the invention provides a chimeric molecule. The chimeric molecule includes (a) a peptide agonist selected from the group consisting of GLP-1 or its analog, GIP or its analogs, Exendin-4 or its analogs, and oxyntomodulin and its analogs; (b) a binding domain capable of binding to Low density lipoprotein receptor-related protein 1 (LRP1), øtransferrin receptor, insulin receptor, or a brain endothelial receptor; and (c) a leptin or its analog.

Some of the particular fusion proteins of the invention include (i) Fc-leptin fusion protein having an amino acid sequence of at least 90%, typically at least 95%, often at least 98%, more often at least 99%, and most often 100% sequence identity to SEQ ID NO:41, SEQ NO: 47, or a combination thereof; (ii) a Fc-leptin fusion protein having an amino acid sequence of at least 90%, typically at least 95%, often at least 98%, more often at least 99%, and most often 100% sequence identity to SEQ ID NO:42, and having at least 1, 2 or 3 glycosylation sites and (iii) a fusion protein having an amino acid sequence of at least 90%, typically at least 95%, often at least 98%, more often at least 99%, and most often 100% sequence identity to SEQ ID NO:43.

Another aspect of the invention provides Fc-Leptin fusion protein. Without being bound by any theory, it is believed such a fusion protein is believed to further increase the in vivo half-life of leptin through introduction of mutations to Fc domain of the fusion protein and increase the binding affinity to Fc receptor FCRN. One particular fusion proteins of the invention includes Fc-leptin fusion protein having an amino acid sequence of at least 90%, typically at least 95%, often at least 98%, more often at least 99%, and most often 100% sequence identity to SEQ ID NO:48.

Yet another aspect of the invention provides a Fc-Leptin fusion molecule that is linked (i.e., fused) to albumin. Without being bound by any theory, it is believed that attaching albumin further increase the in vivo half-life of the Fc-Leptin fusion protein. One particular embodiment of the fusion protein comprising a canine Fc-leptin fusion protein that is linked to a canine albumin has an amino acid sequence of at least 90%, typically at least 95%, often at least 98%, more often at least 99%, and most often 100% sequence identity to SEQ ID NO:49.

Still another embodiment of the invention provides nucleic acid sequences of the canine Fc-Leptin fusion proteins, having a nucleic acid sequence of at least 90%, typically at least 95%, often at least 98%, more often at least 99%, and most often 100% sequence identity to SEQ ID NO:50, 51, or 52.

In another aspect of the invention, a method is provided for expressing the canine Fc-Leptin fusion proteins in a microorganism such as E. coli. Exemplary microorganisms suitable for expressing fusion proteins of the invention include bacteria, yeast, as well as other microoganisms that are known to one skilled in the art. Typically, when expressed using E. coli, the fusion protein is expressed in an inclusion body form. In this manner, the inclusion body is recovered, solubilized and the fusion protein subsequently oxidized and purified to its biologically active form(s).

Another aspect of the invention provides a method of refolding the microorganism (e.g., E. coli) expressed Fc-Leptin fusion proteins. Typically, the refolding of the expressed protein takes place by diluting the solubilized inclusion body into an oxidation solution. In one particular embodiment, the oxidation solution comprises from about 25 mM to about 100 mM of Tris buffer, from about 1 M to about 3 M of urea, from about 5% to about 15% of sucrose, from about 75 mM to about 300 mM ariginine, a redox pair of cysteine at a concentration of from about 0.5 mM to about 10 mM and cystamine at a concentration of from about 0.1 mM to about 2 mM, and/or addition components. The pH of the oxidation solution typically ranges from about pH 7.5 to about pH 10. Generally, the amount of oxidation solution used is about 4 to about 20 times the volume of the solubilized inclusion body solution. Typically, the mixture is allowed to incubate at a temperature from about 0° C. to about 30° C. for from about 4 h to about 48 h.

In one particular embodiment, the oxidation solution comprises about 50 mM Tris, about 10% of sucrose, about 150 mM of arginine, about 2.5 M of urea, about 10 mM of cysteine, about 1 mM of cystamine, and a pH of about pH 9.

Another aspect of the invention provides a fragment antigen-binding (Fab) protein that is linked to a leptin. One particular embodiment of this aspect of the invention is a Fab-leptin fusion molecule comprising a leptin that is linked to a C-terminus of both a heavy chain and a light chain of a Fab protein. In some instances, the heavy chain has an amino acid sequence of at least 90% sequence identity to SEQ ID NO:44. In other instances, the light chain has an amino acid sequence of at least 90% sequence identity to SEQ ID NO:45.

Still another aspect of the invention provide an antibody-leptin fusion molecule comprising a leptin that is linked to a C-terminus of a heavy chain of an antibody. In this aspect of the invention, the antibody comprises a heavy chain and a light chain, where the heavy chain of the antibody has an amino acid sequence of at least 75% sequence identity to SEQ ID NO:46.

Other aspects of the invention include (i) a polynucleotide encoding a fusion protein described herein; (ii) an expression vector comprising such a polynucleotide; and (iii) a host cell transfected with such a vector.

Still other aspects of the invention provide (i) a method of producing a fusion protein or a chimeric molecule described herein, (ii) a pharmaceutical composition comprising a fusion protein or a chimeric molecules described herein, and a pharmaceutically acceptable carrier; and (iii) a method for treating a metabolic disorder in a subject using such a pharmaceutical composition.

### BRIEF DESCRIPTION OF THE DRAWINGS

FIG. 1 is a schematic illustration of a canine Fc-Leptin Analog fusion protein in accordance with the present invention.

FIG. 2 is a schematic illustration of a Fab-Leptin Analog fusion protein. The Fab binds to a brain endothelial receptor.

FIG. 3 is a schematic illustration of an Exendin-4/Antibody/Leptin Analog chimeric molecule. The antibody portion binds to a brain endothelial receptor.

FIG. 4 is a schematic illustration of a Canine IgGB Fc-Leptin Fusion Molecule.

FIG. 5 is a schematic illustration of a Canine IgGB Fc-Leptin Fusion Molecule with Point Mutations at the Fc Domain designed to increase FcRN Binding Affinity.

FIG. 6 is a schematic illustration of a Canine Albumin-IgGB Fc-Leptin Fusion Molecule.

FIG. 7 shows the results of a SDS-PAGE analysis of cell lysate, and inclusion bodies.

FIG. 8 shows the results of a SDS-PAGE analysis of the oxidation pool at different pHs and time points.

FIG. 9 shows the results of a SDS-PAGE analysis of the protein A affinity chromatography purified pool samples.

FIG. 10 shows a cell-based activity assay results of Fc-Leptin Fusion ProteinA.

FIG. 11 shows a cell-based activity assay results of Fc-Leptin Fusion ProteinB and C.

FIG. 12 shows amino acid sequences of leptin and analogs in different species.

FIG. 13 shows amino acid sequences of IgG Fc and analogs in different species.

FIG. 14 shows some of the amino acid sequences of peptides and antibodies or antibody fragments facilitating delivery of the leptin fusion protein across blood-brain barrier (“BBB”).

FIG. 15 shows amino acid sequences of the serum albumin protein for different species.

FIG. 16 is a table showing amino acid sequences of GLP-1, GIP, Oxyntomodulin, Glucagon and analogs. In the table “Aib” refers to aminoisobutyric acid.

FIG. 17 is a table showing illustrative examples of peptide linkers that can be used in the invention.

FIG. 18 shows exemplary amino acid sequences for some of the leptin fusion proteins of the invention.

FIG. 19 shows exemplary nucleic acid sequences for leptin fusion proteins of the invention.

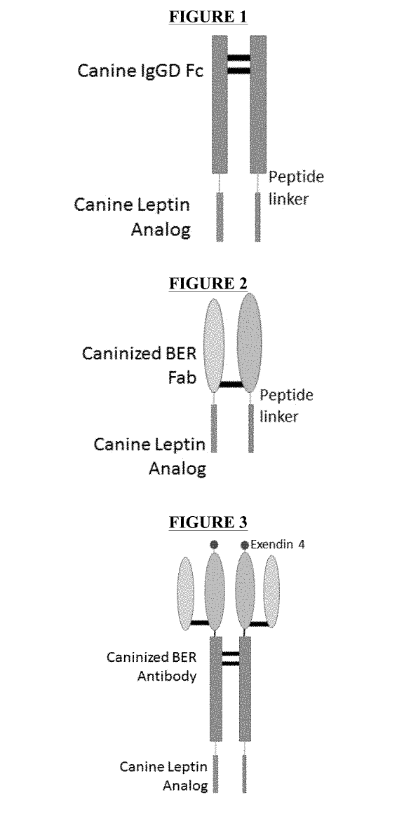

### DETAILED DESCRIPTION OF THE INVENTION

The present invention provides various fusion proteins and chimeric proteins that have at least a portion of a leptin protein amino acid sequence as well as methods for producing and using the same. The present inventors have discovered that by linking an amino acid sequence comprising at least a portion of a leptin to an antibody or a fragment thereof provides improved in vivo half-life and/or increased efficacy of leptin. Typically, the half-life of the fusion protein and chimeric protein of the invention is at least about ten (10) time (i.e., 1,000%) or more, typically at least about thirty (30) times or more, and often at least about one hundred (100) times or more compared to the corresponding native leptin. The term “about” means ±20%, typically ±10%, and often ±5% of the numeric value. The term “corresponding native leptin” refers to the leptin from the same species as that of the fusion proteins or the chimeric proteins of the invention.

One particular aspect of the invention provides a fusion protein comprising a first protein that is linked to a second protein. In some embodiments, the first protein comprises (a) an amino sequence that is at least 75%, typically at least 80%, often at least 85%, more often at least 90%, still more often at least 95%, even more often at least 96%, yet more often at least 97%, still yet more often at least 98%, yet even more often at least 99% and most often 100% sequence identity to a canine immunoglobulin Fc (“Ig Fc”) region, typically IgG Fc region; (b) a canine albumin having amino acid sequence of at least 75%, at least 80%, at least 85%, at least 90%, at least 95%, at least 96%, at least 97%, at least 98%, at least 99% or 100%, sequence identity to SEQ ID NO:25; (c) a feline Ig Fc region; or (d) a feline albumin having amino acid sequence of at least 75%, at least 80%, at least 85%, at least 90%, at least 95%, at least 96%, at least 97%, at least 98%, at least 99% or 100%, sequence identity to SEQ ID NO:26. Depending on the identity of the first protein, the second protein can be either a canine leptin protein or a feline leptin protein. In some embodiments, the first and the second proteins are linked via a linker. The linker can be an oligopeptide, as exemplified in SEQ ID NOS:36-40, or it can be a other protein or peptide linkers known to one skilled in the art, such as, but not limited to, polyglycol linkers, polysaccharide linkers, polyethylene linkers, etc.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection [see generally, Current Protocols in Molecular Biology, (Ausubel, F. M. et al., eds.) John Wiley & Sons, Inc., New York (1987-1999, including supplements such as supplement 46 (April 1999)]. Use of these programs to conduct sequence comparisons are typically conducted using the default parameters specific for each program.

Another example of algorithm that is suitable for determining percent sequence identity and sequence similarity is the BLAST algorithm, which is described by Altschul et al. in J. Mol. Biol., 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to” or “specifically hybridizing to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe or primer will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. In other instances, stringent conditions are chosen to be about 20° C. or 25° C. below the melting temperature of the sequence and a probe with exact or nearly exact complementarity to the target. As used herein, the melting temperature is the temperature at which a population of double-stranded nucleic acid molecules becomes half-dissociated into single strands. Methods for calculating the Tₘ of nucleic acids are well known in the art (see, e.g., Berger and Kimmel (1987) Methods in Enzymology, vol. 152: Guide to Molecular Cloning Techniques, San Diego: Academic Press, Inc. and Sambrook et al. (1989) Molecular Cloning: A Laboratory Manual, 2nd ed., vols. 1-3, Cold Spring Harbor Laboratory), both incorporated herein by reference. As indicated by standard references, a simple estimate of the T., value can be calculated by the equation: Tₘ=81.5+0.41(% G+C), when a nucleic acid is in aqueous solution at 1 M NaCl (see e.g., Anderson and Young, “Quantitative Filter Hybridization,” in Nucleic Acid Hybridization (1985)). Other references include more sophisticated computations which take structural as well as sequence characteristics into account for the calculation of T. The melting temperature of a hybrid (and thus the conditions for stringent hybridization) is affected by various factors such as the length and nature (DNA, RNA, base composition) of the probe or primer and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook, supra, and Ausubel, supra. Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions can also be achieved with the addition of destabilizing agents such as formamide.

In one embodiment, when the first protein is a canine Ig Fc (typically IgG Fc) region or a canine albumin, the second protein is a canine leptin protein having an amino acid sequence that is at least 87%, at least 90%, at least 92%, at least 95%, at least 96%, at least 97%, at least 98%, at least 99% or 100%, sequence identity to a canine leptin or its analog of SEQ ID NO:4 or SEQ ID NO:5, respectively. When the first protein is a feline Ig Fc region or a feline albumin, the second protein is a feline leptin or its analog having an amino acid sequence that is at least 87%, at least 90%, at least 92%, at least 95%, at least 96%, at least 97%, at least 98%, at least 99% or 100%, sequence identity to SEQ ID NO:6 or SEQ ID NO:7, respectively.

The second protein can be linked to a C-terminus or an N-terminus of the first peptide.

In some embodiments, the canine Ig Fc region comprises an amino acid sequence with at least 90%, at least 92%, at least 95%, at least 96%, at least 97%, at least 98%, at least 99% or 100%, sequence identity to SEQ ID NO:11, SEQ ID NO:12, SEQ ID NO:13 or SEQ ID NO:14. In one particular embodiment, the canine Ig Fc region peptide is a canine IgGD Fc region comprising an amino acid sequence with at least 90%, at least 92%, at least 95%, at least 96%, at least 97%, at least 98%, at least 99% or 100%, sequence identity to SEQ ID NO:14.

Still in another embodiment, the canine IgGD Fc region comprises ProAlaAla (S10P/L16A/L17A) mutant of SEQ ID NO:14. Yet in another embodiment, the canine IgGD Fc region comprises 1879Q and M213L mutant of SEQ ID NO:14.

In another embodiment, the feline Ig Fc (typically IgG Fc) region comprises an amino acid sequence having at least 85%, at least 90%, at least 92%, at least 95%, at least 96%, at least 97%, at least 98%, at least 99% or 100%, sequence identity to SEQ ID NO:15 or SEQ ID NO:16.

The fusion protein of the invention can also include a binding domain. The binding domain can be a peptide, an antibody, or an antibody fragment. The binding domain is capable of binding to Low density lipoprotein receptor-related protein 1 (LRP1), transferrin receptor, insulin receptor, or a brain endothelial receptor, thereby resulting in endocytosis or transcytosis of a receptor. Moreover, the presence of a binding domain increases delivery of the fusion protein through a blood brain barrier.

In one embodiment, the binding domain is a Angiopep-2 peptide selected from the group consisting of SEQ ID NO:17 and SEQ ID NO:18. In some instances, the Angiopep-2 peptide is linked to an N-terminus of the fusion protein.

Yet in another embodiment, the binding domain comprises an amino acid sequence having at least 75%, at least 80%, at least 85%, at least 90%, at least 95%, at least 96%, at least 97%, at least 98%, at least 99% or 100% sequence identity to SEQ ID NO:19. Other suitable binding domain include those having an amino acid sequence of at least 75%, at least 80%, at least 85%, at least 90%, at least 95%, at least 96%, at least 97%, at least 98%, at least 99% or 100% sequence identity to any one of SEQ ID NOS:20-24.

The fusion protein can also include a peptide agonist that is capable of activating a receptor selected from the group consisting of: (a) GLP-1 receptor; b) Gastric Inhibitory Polypeptide (GIP) receptor, (c) Glucagon receptor, and (d) a combination of two or more thereof. The peptide agonist can be linked to the fusion protein by a linker. Suitable linkers include those discussed herein. In one particular embodiment, the peptide agonist is selected from the group consisting of (i) GLP-1 or its analogs; (ii) exendin-4 or its analog; (iii) GIP or its analogs; and (iv) Oxyntomodulin or its analogs. The peptide agonist can be linked to an N-terminus or the C-terminus of the fusion protein. In another embodiment, the peptide agonist comprises an amino acid sequence selected from the group consisting of SEQ ID NOS:28-35.

Another aspect of the invention provides a fusion protein comprising as a first protein a human leptin or its analog with an amino acid sequences with at least 85%, at least 90%, at least 92%, at least 95%, at least 96%, at least 97%, at least 98%, at least 99% or 100%, sequence identity to SEQ ID NO:1, SEQ ID NO:2 or SEQ ID NO:3; and a second protein that is linked to the first protein. The second protein comprises a Fc fragment selected from the group consisting of human IgG1 Fc, human IgG2 Fc and human IgG4 Fc, e.g., those having an amino acid sequence of at least 75%, at least 80%, at least 85%, at least 90%, at least 95%, at least 96%, at least 97%, at least 98%, at least 99% or 100% sequence identity to SEQ ID NOS:8-10, respectively. In some embodiments, the second protein is a human albumin having amino acid sequence of at least 75%, at least 80%, at least 85%, at least 90%, at least 95%, at least 96%, at least 97%, at least 98%, at least 99% or 100%, sequence identity to SEQ ID NO:27. In some embodiments, the first and the second proteins are linked via a linker. The linker can be an oligopeptide, as exemplified in SEQ ID NOS:36-40, or it can be a other protein or peptide linkers known to one skilled in the art, such as, but not limited to, polyglycol linkers, polysaccharide linkers, polyethylene linkers, etc. Within this aspect of the invention, the fusion protein can also include a peptide agonist that is capable of activating a receptor selected from the group consisting of: (a) GLP-1 receptor; b) Gastric Inhibitory Polypeptide (GIP) receptor, (c) Glucagon receptor, and (d) a combination of two or more thereof. In one particular embodiment, the peptide agonist comprises amino acid sequence selected from the group consisting of SEQ ID NOS:28-35.

Still another aspect of the invention provides a chimeric molecule. The chimeric molecule of the invention includes (a) a peptide agonist selected from the group consisting of GLP-1 or its analog, GIP or its analogs, Exendin-4 or its analogs, and oxyntomodulin and its analogs; (b) a binding domain capable of binding to Low density lipoprotein receptor-related protein 1 (LRP1), transferrin receptor, insulin receptor, or a brain endothelial receptor; and (c) a leptin or its analog. The chimeric molecule can also include a linker between the peptide agonist and the binding domain. In addition, or alternatively, the chimeric molecule can also include a linker between the binding domain and leptin.

In one embodiment, the leptin in the chimeric molecule includes an amino acid sequence having at least 75%, at least 80%, at least 85%, at least 90%, at least 95%, at least 96%, at least 97%, at least 98%, at least 99% or 100%, sequence identity to one of SEQ ID NOS:1-7.

In some embodiments, the peptide agonist of the chimeric molecule includes an amino acid sequence selected from the group consisting of SEQ IDS:28-35.

Yet in other embodiments, the binding domain of the chimeric molecule comprises an amino acid sequence of a Agiopep-2 peptide selected from the group consisting of SEQ ID NO:17 and SEQ ID NO:18.

Still in other embodiments, the binding domain of the chimeric molecule is an antibody or an antibody fragment. The antibody or the antibody fragment is capable of binding to Low density lipoprotein receptor-related protein 1 (LRP1), transferrin receptor, insulin receptor, or a brain endothelial receptor. In one particular embodiment, the binding domain comprises an amino acid sequence having at least 75%%, at least 80%, at least 85%, at least 90%, at least 95%, at least 96%, at least 97%, at least 98%, at least 99% or 100%, sequence identity to SEQ ID NOS:19 or 20.

Specific examples of fusion proteins and/or chimeric molecules of the invention include, but are not limited to: (i) Fc-leptin fusion protein having an amino acid sequence of at least 90%, at least 95%, at least 98%, at least 99%, or 100% sequence identity to SEQ ID NO:41 and SEQ NO: 47, (ii) a Fc-leptin fusion protein having an amino acid sequence of at least 90%, at least 95%, at least 98%, at least 99%, or 100% sequence identity to SEQ ID NO:42, and having at least 1, 2 or 3 glycosylation sites and (iii) a fusion protein having an amino acid sequence of at least 90%, at least 95%, at least 98%, at least 99%, or 100% sequence identity to SEQ ID NO:43.

Another aspect of Fc-Leptin fusion protein is to further increase (i) the in vivo half-life of leptin through introduction of mutations to Fc domain of the fusion protein (ii) and/or the binding affinity to Fc receptor FCRN. One of the particular fusion proteins of the invention includes Fc-leptin fusion protein having an amino acid sequence of at least 90%, at least 95%, at least 98%, at least 99%, or 100% sequence identity to SEQ ID NOS:47 or 48. One specific fusion protein of the invention includes Fc-leptin fusion protein having an amino acid sequence of at least 90%, at least 95%, at least 98%, at least 99%, or 100% sequence identity to SEQ ID NO:48.

In some embodiments, the Fc-Leptin fusion protein is fused (i.e., linked or attached) to albumin. Without being bound by any theory, it is believed this further increases in vivo half-life of Fc-Leptin protein. One particular fusion proteins of the invention comprises a canine Fc-leptin fusion protein linked to canine albumin. Exemplary Fc-leptin fusion protein linked to canine albumin include an amino acid sequence having at least 90%, at least 95%, at least 98%, at least 99%, or 100% sequence identity to SEQ ID NO:49.

Still other embodiments of the invention include nucleic acid sequences of the canine Fc-Leptin fusion proteins having at least 90%, at least 95%, at least 98%, at least 99%, or 100% sequence identity to SEQ ID NO:50, 51, or 52.

Another aspect of the invention provides method of producing the canine Fc-Leptin fusion proteins using a host cell. While the host cell can be any microorganism known to one skilled in the art, one particular host cell used in producing Fc-leptin fusion protein is E. coli. In some embodiments, the fusion protein is expressed in the inclusion body form. The method can also include recovering the inclusion body, solubilizing and oxidizing the fusion protein to produce a biologically active fusion protein, which can be purified.

The E. coli expressed canine Fc-Leptin fusion proteins can be refolded to obtain a biologically active form. Typically, the refolding of the fusion protein is achieved by diluting the solubilized inclusion body into an oxidation solution. A typical oxidation solution comprises Tris at 25-100 mM, urea at 1-3 M, sucrose at 5-15%, ariginine at 75-300 mM, a redox pair of cysteine at a concentration of 0.5-10 mM and cystamine at a concentration of 0.1 to 2 mM, and/or addition components, and at a pH of 7.5 to 10. Generally, the amount of oxidation solution used is about 4-20 times the volume of the solubilized inclusion body solution. The mixture typically is allowed to incubate at 0-30° C. for 4-48 hours. In one particular embodiment, the oxidation solution comprises about 50 mM Tris, about 10% sucrose, about 150 mM arginine, about 2.5 M urea, about 10 mM cysteine, about 1 mM cystamine, and at a pH of about pH 9.

Yet another aspect of the invention provides a Fab-leptin fusion molecule comprising a leptin that is linked to a C-terminus of both a heavy chain and a light chain of a fragment antigen-binding (Fab) protein. The heavy chain has an amino acid sequence of at least 90%%, at least 95%, at least 96%, at least 97%, at least 98%, at least 99% or 100% sequence identity to SEQ ID NO:44. The light chain has an amino acid sequence of at least 90%%, at least 95%, at least 96%, at least 97%, at least 98%, at least 99% or 100% sequence identity to SEQ ID NO:45.

Still yet another aspect of the invention provides an antibody-leptin fusion molecule comprising a leptin that is linked to a C-terminus of a heavy chain of an antibody. The antibody comprises a heavy chain and a light chain. The heavy chain of the antibody has an amino acid sequence of at least 75%%, at least 80%, at least 85%, at least 90%, at least 95%, at least 96%, at least 97%, at least 98%, at least 99% or 100% sequence identity to SEQ ID NO:46.

Other aspects of the invention include (i) a polynucleotide encoding the fusion protein or the chimeric molecule disclosed herein, (ii) an expression vector comprising such a polynucleotide, and (iii) a host cell transfected with such a vector. Suitable polynucleotide sequences encoding the fusion proteins of the invention include, but are not limited to, those shown in SEQ ID NOs:50, 51 and 52. The term “expression vector” means a linear or circular DNA molecule that comprises a polynucleotide encoding a polypeptide and is operably linked to control sequences that provide for its expression. The term “host cell” means any cell type that is susceptible to transformation, transfection, transduction, or the like with a nucleic acid construct or expression vector comprising a polynucleotide of the present invention. The term “host cell” also encompasses any progeny of a parent cell that is not identical to the parent cell due to mutations that occur during replication. Typical host cells are microorganisms such as bacteria or yeast. Often E. coli is used as a host cell for transcription. However, it should be appreciated that the scope of the invention is not limited to E. coli as one skilled in the art can readily recognize a suitable host cells for transcription of the expression vector disclosed herein. The term “isolated” or “recovered” means a substance in a form or environment that does not occur in nature.

One particular method for producing a biologically active fusion protein disclosed herein include (i) culturing a host cell under conditions sufficient to produce a fusion protein from the expression vector in an inclusion body form; and oxidizing said fusion protein under conditions sufficient to produce a biologically active fusion protein. In some embodiments, the oxidizing step comprises: (i) obtaining said inclusion body from said host cell; (ii) solubilizing obtained inclusion body in the presence of an oxidizing agent under conditions sufficient to produce said biologically active fusion protein; and (iii) optionally purifying said biologically active fusion protein.

The present invention also includes a pharmaceutical composition comprising a fusion protein or a chimeric molecules described herein. The pharmaceutical composition of the invention can also include a pharmaceutically acceptable carrier.

The pharmaceutical composition of the invention can be used to treat a metabolic disorder in a subject, such as human, a dog or a cat. In one particular embodiment, the metabolic disorder is selected from the group consisting of obesity, diabetes, a heart disease (e.g., atherosclerosis), and a liver disease (e.g. fatty liver disease).

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1

Expression and Purification of Leptin Fusion Protein by CHO Cells. DNA for the chimeric molecule comprising the Fc-leptin fusion protein (SEQ ID NO: 42, named as ASKB-O42) is synthesized and cloned into a bacterial expression vector. The complete expression construct comprising the DNA gene is confirmed by DNA sequencing. The expression construct is amplified by transforming into DH10B E. coli and culturing the cells overnight. DNA for the expression construct was prepared and purified by endo-free plasmid kit (from)QIAGEN®.

Cell lines stably expressing ASKB-O42 is obtained by transfecting the expression construct into GS^{-/-} Chinese hamster ovarian cells (CHO) by electroporation and screening for transfected CHO cells using a selective culture medium without glutamine (EX-CELL® CD CHO Fusion Growth Medium). In this manner 32 or more stable minipools are established and the leading mini-pool is selected based on expression level in batch and fed-batch cultures. The expression levels are detected by ELISA titer assay. Single cloning is performed by limited dilution and using clone media, two leading single clones out of more than100 positive clones are selected based on productivity and cell growth in batch and fed-batch culture. The lead clones are expanded and seeded at 0.5×10⁶ cells/mL, total 300 mL in 2 L shake flasks, and the cells are cultured at 37° C., 5% CO₂, 70% HMR conditions and shaking at 120 rpm. The cultures are fed by using 5% Acti CHO® Feed A+0.5% Feed B (from GE Health) on Day 3, 6, 7, 8 and 9. The cell viability, viable cell density are monitored every other day, the cultures are harvested on Day 11-13.

The cell culture medium is harvested by clarifying approximately 600 mL of the cultured cell medium through centrifugation at 2000 rpm for 10 minutes followed by filtration. The clarified supernant is loaded to a Protein A affinity column and the chimeric molecule is purified. The protein is further purified using ion exchange chromatography, hydrophobic interaction chromatography, hydroxyapatite chromatography, and/or mixed mode chromatography. The product is further concentrated and buffer exchanged using UFDF and further formulated. The purity of the product is analyzed using CE-SDS and HPLC methods.

### Example 2

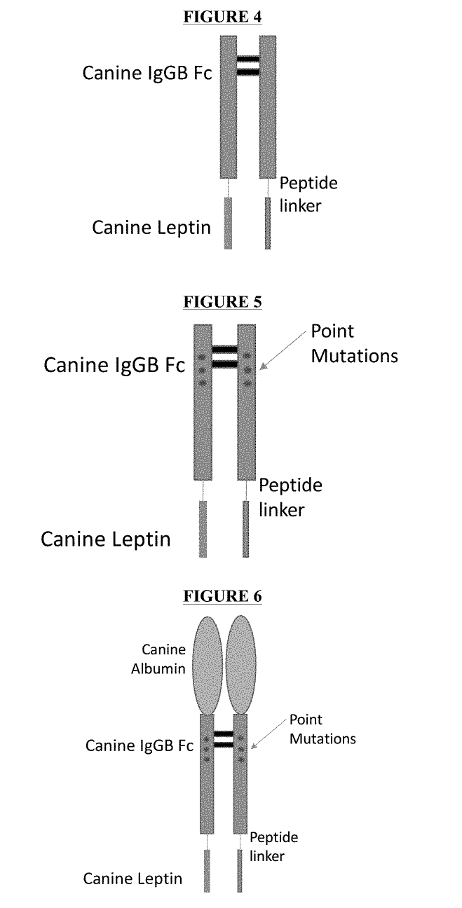

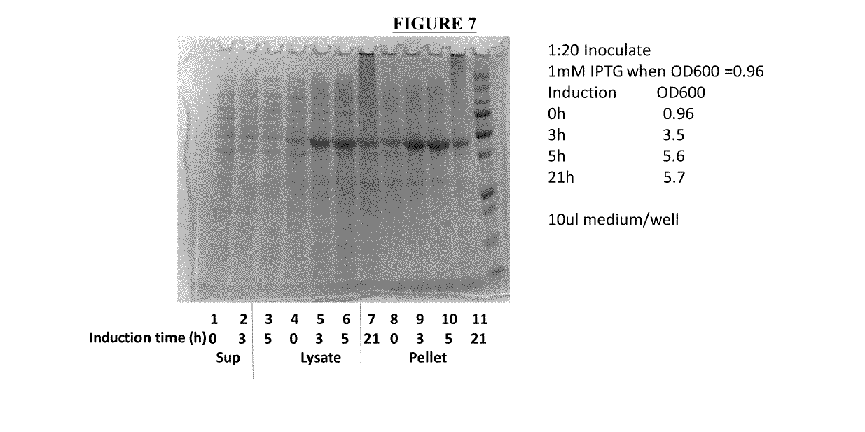

Expression Fc-Leptin Fusion Protein by E. coli. Expression of Fc-Leptin Fusion Proteins A, B and C was carried out by E. coli BL21 DE3 strain. The schematic structures of the Fc-Leptin Fusion Protein A, B and C are illustrated in FIGS. 4, 5, and 6. Plasmids contained the gene sequences as shown in SEQ ID NO: 50, and 52 were synthesized by DNA 2.0. Plasmid containing the gene for the Fc-Leptin Fusion Protein B has a sequence as shown in SEQ ID NO: 51, which was mutated from SEQ ID NO: 50 (Fc-Leptin Fusion Protein A). The E. coli was transformed, plated and positive clones were selected. The overexpression in shake flask was carried out using LB culture medium and the expression was induced with 1 mM IPTG. The cells were harvested after approximately 5 hours to overnight after induction. FIG. 7 shows the expression levels of the Fc-Leptin Fusion Protein A at different time point after IPTG induction. The results indicated that the expression level plateaued at approximately 5 hours after IPTG induction.

### Example 3

Harvest of Inclusion Bodies. Cell paste of approximately 15 grams (wet weight) was resuspended in approximately 60 ml of distilled water. The mixture was sonicated by a Model FB50 sonicator from Fisher Scientific at an amplitude of approximately 85 for 20-30 seconds on ice, three times, with 1 minute in between each of the sonication. The resulted cell lysate was centrifuged for 20 minutes at 3000 RPM using a Sorvall RC 3BP centrifuge. The pellet was washed twice by being resuspended in 60 ml of distilled water and centrifuged. The resulted pellet from the third centrifugation containing the inclusion bodies of the fusion protein was directly processed or stored at −80° C. until further processing.

### Example 4

Solubilization, Refolding and Purification of Fc-Leptin Fusion Protein Expressed in E. coli. The inclusion body (thawed if stored frozen) was solubilized by addition of approximately 50 mM Tris Base, 1.5 M guanindine-HCl, 6 M urea, and 8 mM dithiolthreitol (DTT). The mixture was vigorously mixed and allowed to incubate at room temperature for over 60 minutes.

### Example 5

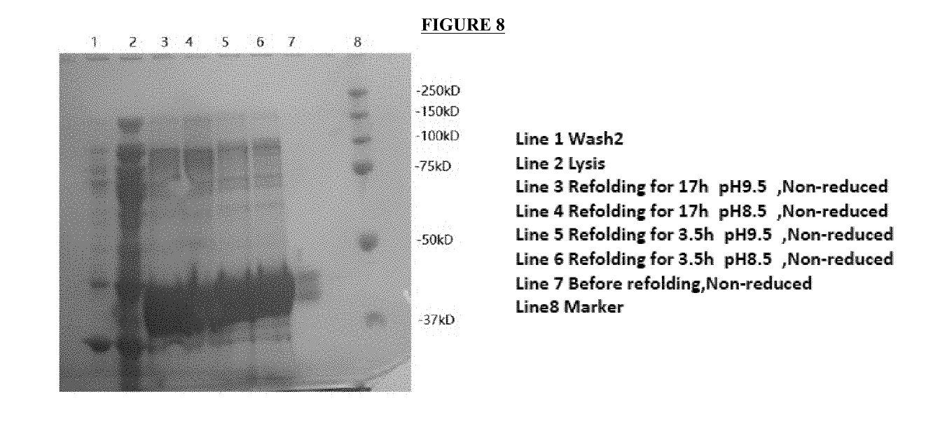

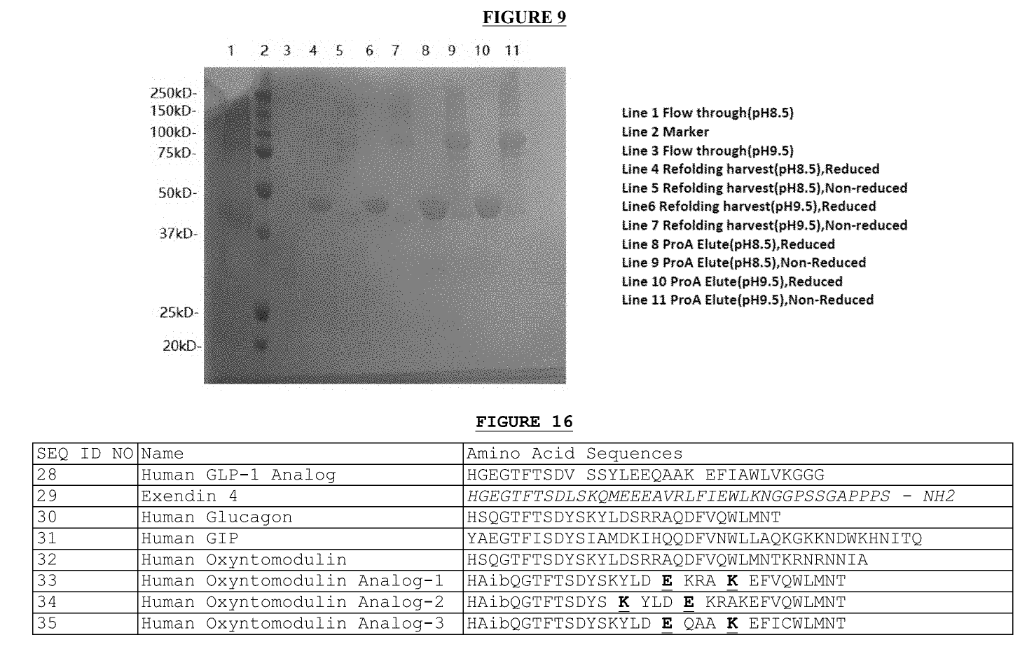

Oxidation and Protein A Purification of the Fc-Leptin Fusion Protein. Approximately 40 ml of the solubilized inclusion body solution was diluted into 210 ml of the oxidation buffer, mixed well, and incubated at 2-8° C. overnight. The oxidation pool was clarified and loaded on to a 10 ml Protein A affinity column. The column was washed and eluted with 50 mM acetic acid, pH 3.6. The Protein A pool was titrated to pH approximately 5 and filtered. The purity of the Protein A pool was analyzed by SDS-PAGE and SEC-HPLC. The activity of the Protein A pool samples was also analyzed by a cell-based activity assay. FIG. 8 shows the analysis of the oxidation pool at pH 8.5 and 9.5 and at different time points. The results indicated that dimers were formed at both pHs after 17 hours of incubation. FIG. 9 shows the results of the SDS-PAGE analysis of the Fc-Leptin Fusion Protein A after Protein A Affinity Purification.

### Example 6

In Vitro Biological Activity Study: HEK 293 cells were stably transfected with both the luciferase reporter gene under control of a STAT3 response element and the OB-Rb (leptin receptor), which is expressed on the cell surface. Leptin binds to the leptin receptor and activates STAT3 homodimers and STAT3/STAT1 heterodimers, which interact and bind with the STAT3 sequence response element. This interaction drives expression of the luciferase gene and stimulates cells to produce luciferase. After addition of the luciferase substrate and reaction, the amount of luminescence is proportional to the activity of the compound. The biological activity is based on the EC50 of a 4-PL sigmoidal curve.

DAY ONE: Cells were seeded into a 96-well plate at 30,000 cells/50 microL/well and placed in a 37° C., 5% C02 incubator for overnight.

DAY TWO: The starting concentration for wild type (WT) canine leptin was 100 ng/ml and the Fc-leptin fusion protein of 1 to 400 ng/mL. From that, 50 μL volume was taken from each sample and 3× serial dilutions were made across the rows of the dilution block. Then 50 μL was transferred to each well of the assay plate. After 6 hours of incubation, 100 μL of the luciferase substrate was added to each well. The plate is then read on a Biotek Synergy HTX plate reader.

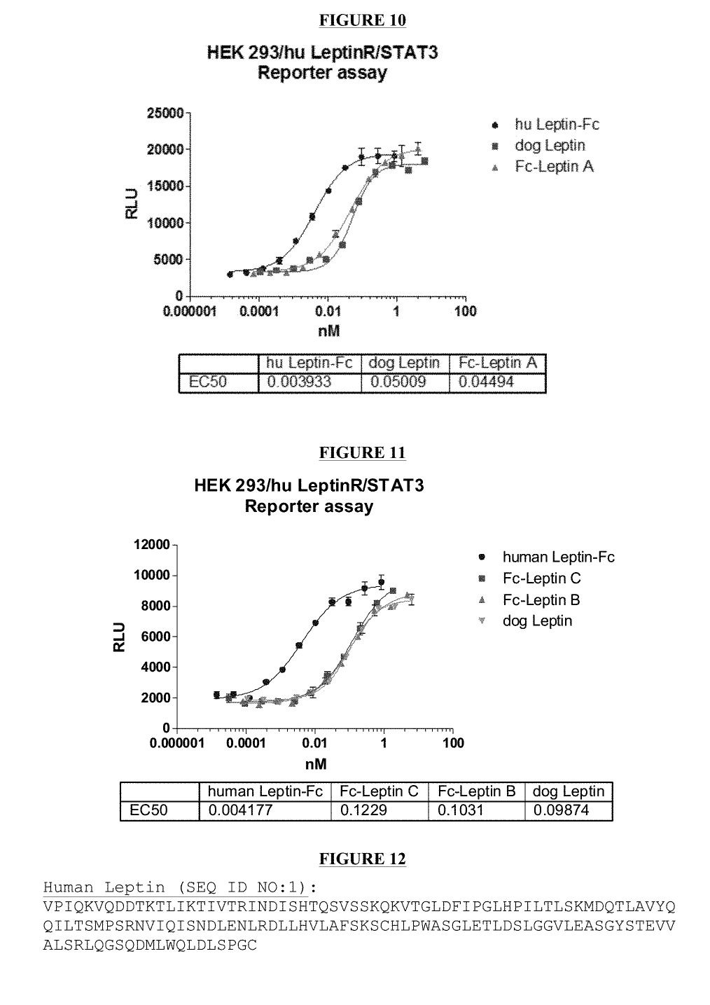

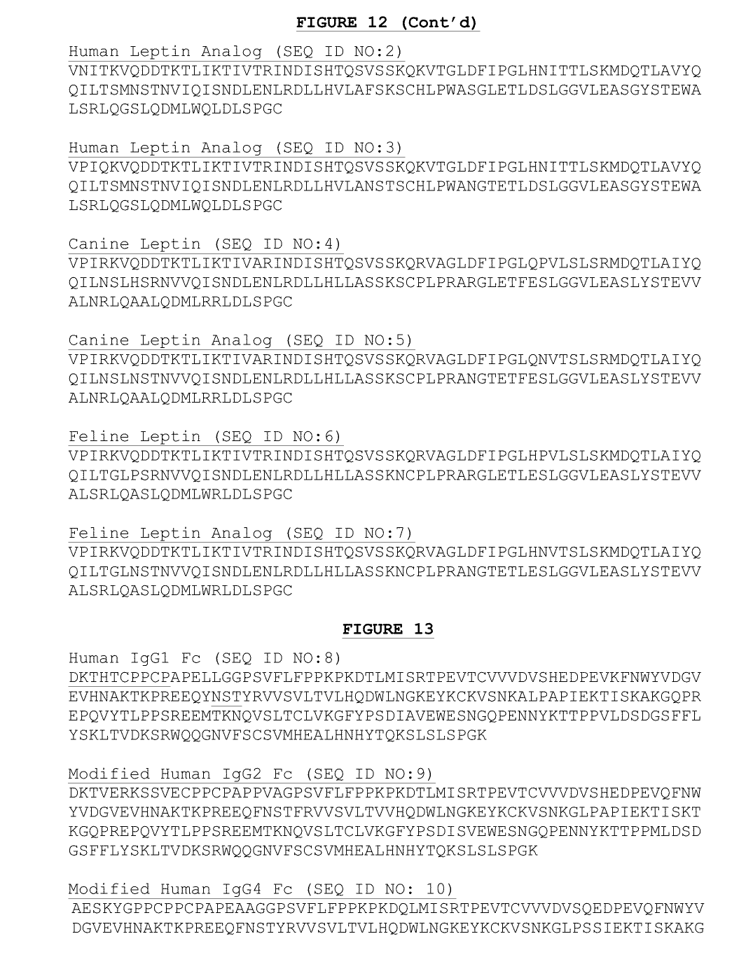

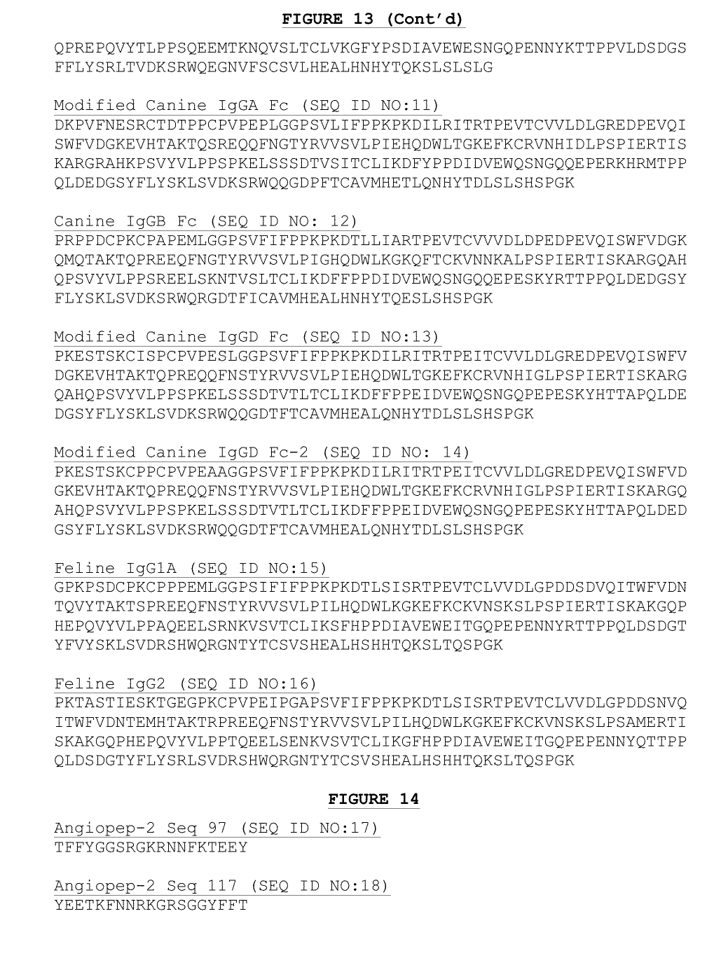

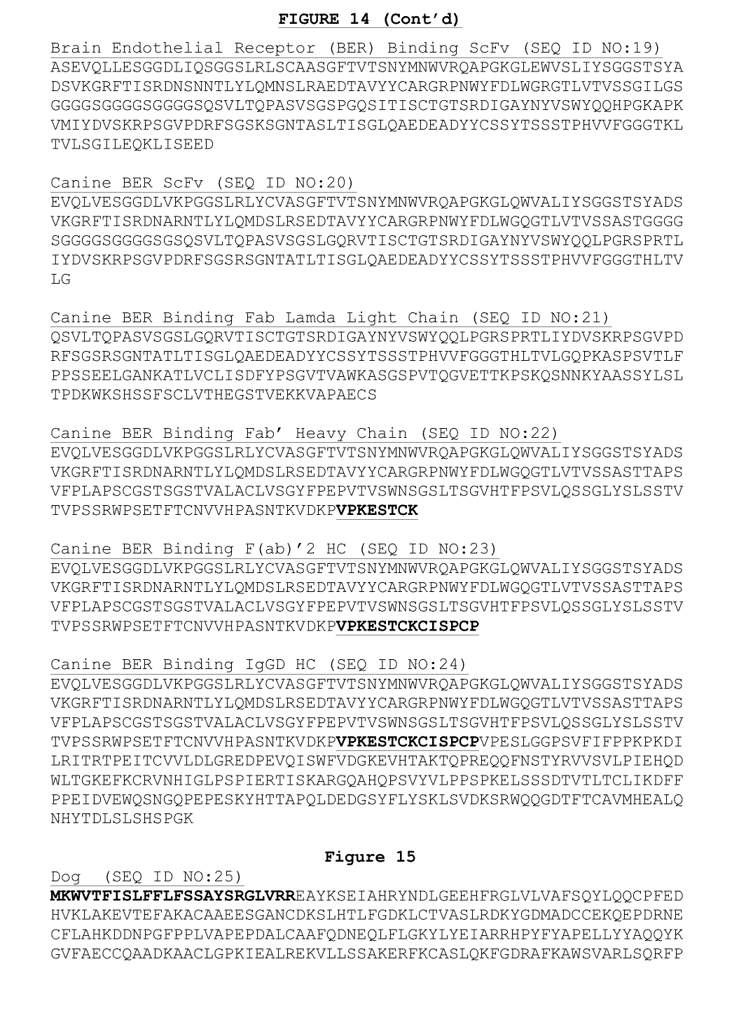

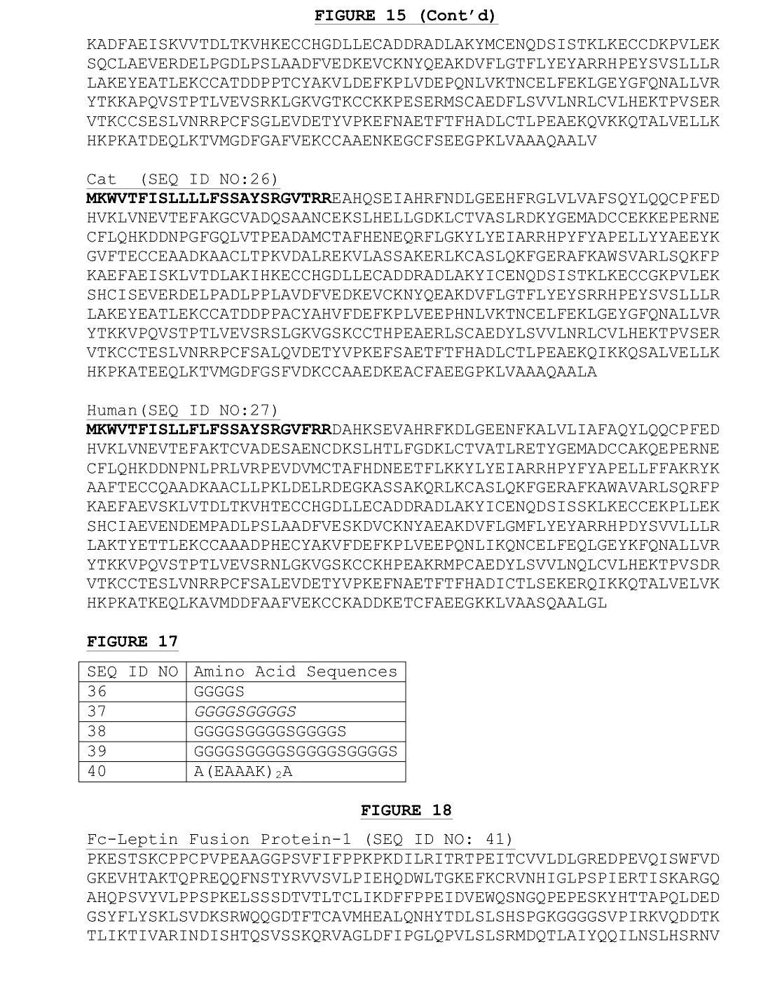

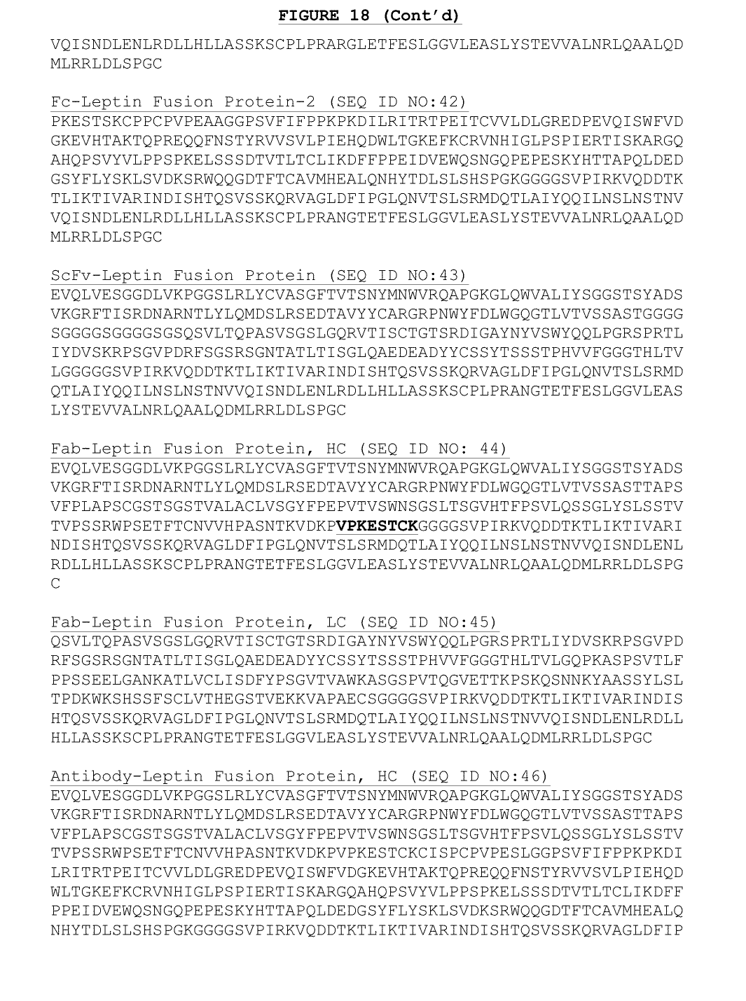

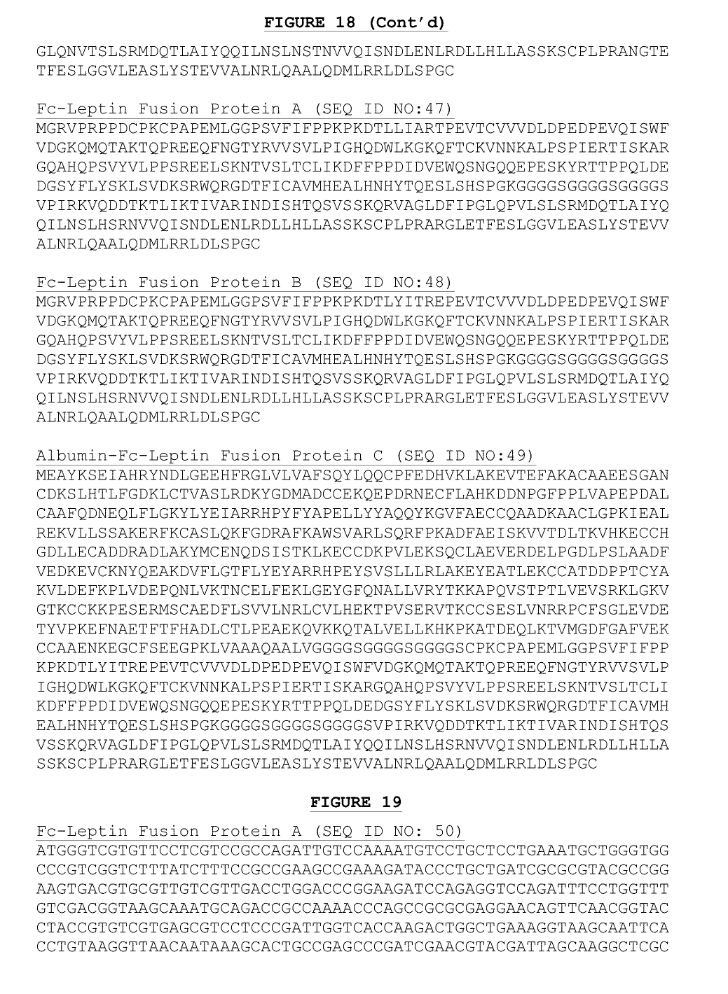

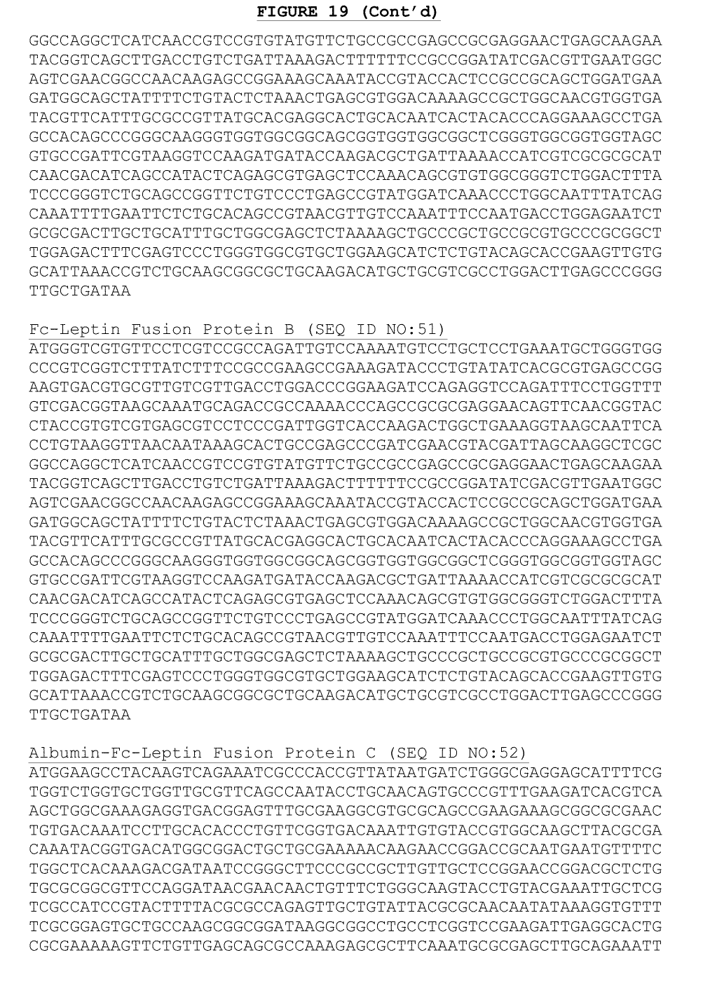

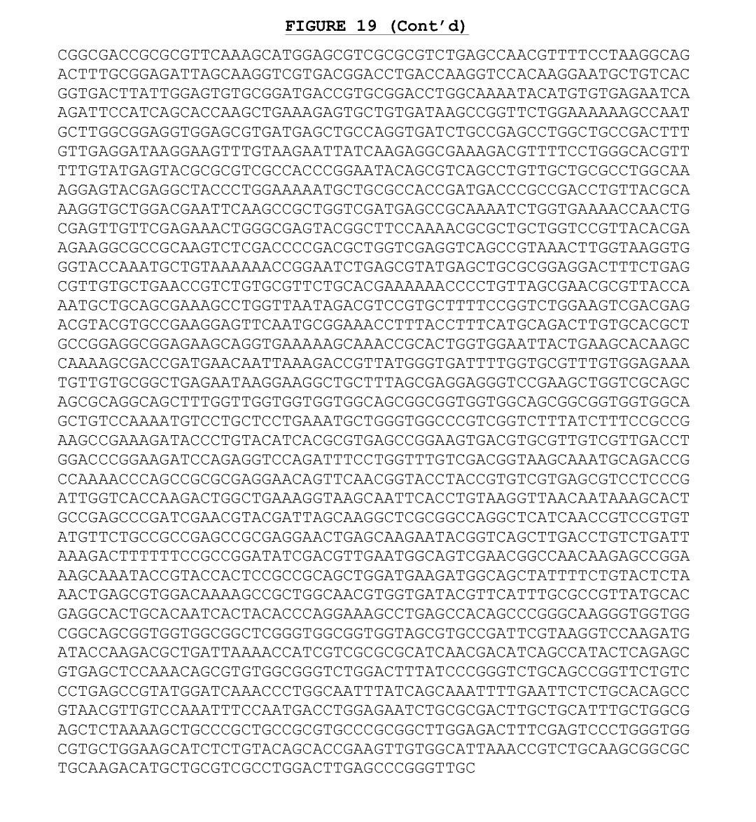

FIG. 10 show the cell-based activity of the Fc-Leptin Fusion Protein A. It showed that the EC₅₀ was similar as that of canine leptin. FIG. 11 show the cell-based activity of the Fc-Leptin Fusion Protein B and C. It showed that the activities of the Fc-Leptin B and C were similar to that of canine leptin.

### Example 7

In vivo Biological Activity Study: The objective of this study is to evaluate the impact of the ASKB-O42 upon body weight, body composition, and feeding behavior in obese male and female dogs. The ASKB-O42 of Example 1 formulated at 5.1 mg/ml in phosphate buffered saline with a pH of 7.4 and 4% (w/v) trehalose.

Eighteen (18) dogs all over one (1) year old are used: nine (9) intact male and nine (9) intact female beagles. The dogs are obese, weighing approximately 12 to 18 kg (26.4 to 39.6 lbs). During the first four weeks of the acclimation period, (or until the desired starting weight is achieved), dogs are fed a laboratory a high fat (approx. 45%) dry food that meets or exceeds the nutritional requirements for maintenance and health. All dogs are fed ad libitum, during this portion of the acclimation period to facilitate weight gain. During the last two weeks of the acclimation period and for the remainder of the study, all dogs are fed a commercial normal fat (approx. 12%) dry dog food, in order to reduce endogenous leptin levels and restore leptin sensitivity. Dogs in treatment groups 1 and 2 continue to be provided with food ad libitum throughout the remainder of the study. Animals in treatment group 3 are fed twice per day according to the label instructions for the diet. Animals are allowed ad libitum access to water via bowls or an automatic watering system contained in each cage. No other concomitant medications are administered during the course of the study.

This study is conducted as a randomized block design within each gender. Dogs are randomly assigned to pens. Animals are blocked by baseline (Day—14) bodyweights within each gender. There are three blocks with three males and three blocks with three females. Block one of males consist of the 3 males with lowest bodyweights and block one of females will consist of the 3 females with lowest bodyweights. The second blocks within each gender consist of the 3 males and 3 females with the next lowest bodyweights. The final block within each gender contains the 3 males and 3 females with the highest bodyweights.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A fusion protein comprising a first protein that is linked to a second protein, wherein said first protein comprises:
(a) a canine immunoglobulin Fc (“Ig Fc”) region;
(b) a canine albumin having amino acid sequence of at least 75% sequence identity to SEQ ID NO:25;
(c) a feline Ig Fc region; or
(d) a feline albumin having amino acid sequence of at least 75% sequence identity to SEQ ID NO:26; and
when said first protein is said canine Ig Fc region or said canine albumin, then said second protein is a canine leptin protein having at least 87% amino acid sequence identity with a canine leptin of SEQ ID NO:4 or SEQ ID NO:5; and
when said first protein is said feline Ig Fc region or said feline albumin, then said second protein is a feline leptin having amino acid sequence of at least 87% sequence identity to SEQ ID NO:6 or SEQ ID NO:7.

2. The fusion protein of claim 1, wherein said first protein is linked to said second protein through a protein linker.

3. The fusion protein of claim 1, wherein said second protein is linked to a C-terminus of said first peptide.

4. The fusion protein of claim 1, wherein said canine Ig Fc region comprises an amino acid sequence with at least 90% sequence identity to SEQ ID NO:11, SEQ ID NO:12, SEQ ID NO:13 or SEQ ID NO:14.

5. The fusion protein of claim 4, wherein said canine Ig Fc region peptide is a canine IgGD Fc region comprising an amino acid sequence with at least 90% sequence identity to SEQ ID NO:14, and wherein said canine IgGD Fc region comprises ProAlaAla (S10P/L16A/L17A) mutant of SEQ ID NO:14.

6. The fusion protein of claim 1, wherein said fusion protein has at least 98% sequence identity to SEQ ID NO: 47, 48 or 49.

7. The fusion protein of claim 1, wherein said feline Ig Fc region comprises an amino acid sequence with at least 95% sequence identity to SEQ ID NO:15 or SEQ ID NO:16.

8. The fusion protein of claim 1 further comprising a binding domain, wherein said binding domain is a peptide, an antibody, or an antibody fragment, and wherein said binding domain is capable of binding to Low density lipoprotein receptor-related protein 1 (LRP1), transferrin receptor, insulin receptor, or a brain endothelial receptor, thereby resulting in endocytosis or transcytosis of a receptor and said fusion protein and increasing delivery of said fusion protein through a blood brain barrier.

9. The fusion protein of claim 8, wherein said binding domain is a Angiopep-2 peptide selected from the group consisting of SEQ ID NO:17 and SEQ ID NO:18.

10. The fusion protein of claim 9, wherein said Angiopep-2 peptide is linked to an N-terminus of said fusion protein.

11. The fusion protein of claim 8, wherein said binding domain comprises an amino acid sequence with at least 95% sequence identity to SEQ ID NO:19.

12. The fusion protein of claim 1 further comprising a peptide agonist that is capable of activating a receptor selected from the group consisting of: (a) GLP-1 receptor; b) Gastric Inhibitory Polypeptide (GIP) receptor, (c) Glucagon receptor, and (d) a combination of two or more thereof.

13. The fusion protein of claim 12, wherein said peptide agonist is linked to said fusion protein by a linker.

14. The fusion protein of claim 12, wherein said peptide agonist is selected from the group consisting of (i) GLP-1 or its analogs; (ii) exendin-4 or its analog; (iii) GIP or its analogs; and (iv) Oxyntomodulin or its analogs.

15. The fusion protein of claim 12, wherein said peptide agonist is linked to an N-terminus of said fusion protein.

16. The fusion protein of claim 12, wherein said peptide agonist comprises an amino acid sequence selected from the group consisting of SEQ ID NOS:28-35.

17. A fusion protein comprising as a first protein a human leptin or its analog with an amino acid sequences with at least 85% sequence identity to SEQ ID NO:1, SEQ ID NO:2 or SEQ ID NO:3; and a second protein that is linked to said first protein, wherein said second protein comprises a Fc fragment selected from the group consisting of human IgG1 Fc, human IgG2 Fc and human IgG4 Fc.

18. The fusion protein of claim 17 further comprising a peptide agonist that is capable of activating a receptor selected from the group consisting of: (a) GLP-1 receptor; b) Gastric Inhibitory Polypeptide (GIP) receptor, (c) Glucagon receptor, and (d) a combination of two or more thereof.

19. The fusion protein of claim 18, wherein said peptide agonist comprises amino acid sequence selected from the group consisting of SEQ ID NOS:28-35.

20. A chimeric molecule comprising:
(a) a peptide agonist selected from the group consisting of GLP-1 or its analog, GIP or its analogs, Exendin-4 or its analogs, and oxyntomodulin and its analogs;
(b) a binding domain capable of binding to Low density lipoprotein receptor-related protein 1 (LRP1), transferrin receptor, insulin receptor, or a brain endothelial receptor; and
(c) a leptin or its analog.

21. The chimeric molecule of claim 20 further comprising a linker between said peptide agonist and said binding domain.

22. The chimeric molecule of claim 20 further comprising a linker between said binding domain and said leptin.

23. The chimeric molecule of claim 20, where said leptin comprises an amino acid sequence having at least 75% sequence identity to an amino acid sequence selected from the group consisting of SEQ ID NOS:1-7.

24. The chimeric molecule of claim 20, wherein said peptide agonist comprises an amino acid sequence selected from the group consisting of SEQ IDS:28-35.

25. The chimeric molecule of claim 20, where said binding domain comprises an amino acid sequence selected from the group consisting of SEQ ID NO:17 and SEQ ID NO:18.

26. The chimeric molecule of claim 20, wherein said binding domain is an antibody or an antibody fragment that is capable of binding to Low density lipoprotein receptor-related protein 1 (LRP1), transferrin receptor, insulin receptor, or a brain endothelial receptor.

27. The chimeric molecule of claim 20, wherein said binding domain comprises an amino acid sequence having at least 75% sequence identity to SEQ ID NOS:19 or 20.

28. A Fc-leptin fusion protein selected from the group consisting of:
an amino acid sequence having at least 90% sequence identity to SEQ ID NO:41;
an amino acid sequence having at least 90% sequence identity to SEQ ID NO:42, and having at least 1, 2 or 3 glycosylation sites;
an amino acid sequence having at least 90% sequence identity to SEQ ID NO:43; and
a leptin that is linked to a C-terminus of both a heavy chain and a light chain of a fragment antigen-binding (Fab) protein, wherein said heavy chain has an amino acid sequence of at least 90%, at least 95%, at least 98%, or 100% sequence identity to SEQ ID NO:44; and said light chain has an amino acid sequence of at least 98% sequence identity to SEQ ID NO:45.

29. An antibody-leptin fusion molecule comprising a leptin that is linked to a C-terminus of a heavy chain of an antibody, wherein a heavy chain of said antibody has an amino acid sequence of at least 98% sequence identity to SEQ ID NO:46.

30. A polynucleotide sequence selected from the group consisting of SEQ ID NOs:50, 51 and 52.

31. An expression vector comprising a polynucleotide of claim 30.

32. A host cell transfected with the vector of claim 31.

33. The host cell of claims 32, wherein said host cell is E. coli.

34. A method for producing a biologically active fusion protein, said method comprising:
culturing a host cell of claim 33 under conditions sufficient to produce a fusion protein from the expression vector in an inclusion body form; and
oxidizing said fusion protein under conditions sufficient to produce a biologically active fusion protein.

35. The method of claim 34, wherein said oxidizing step comprises:
at least partially separating said inclusion body from said host cell;
solubilizing said separated inclusion body under denaturing and reducing conditions to produce a denatured fusion protein;
oxidizing and refolding said denatured fusion protein under conditions sufficient to produce a biologically active fusion protein; and
optionally purifying said biologically active fusion protein.

36. The method of claim 35, wherein said step of oxidizing and refolding comprises admixing said denatured fusion protein with an oxidation solution at a volume ratio ranging from about 1:4 to about 1:20.

37. The method of claim 36, wherein said oxidation solution comprises urea at a concentration of from about 1 M to about 3 M, sucrose at a concentration from about 5% to about 15%, ariginine at a concentration from about 75 mM to about 300 mM, and at a pH of about pH 7.5 to about pH 10.

38. The method of claim 37, wherein said admixture is incubated at a temperature ranging from about 0 to about 30° C. for about 4 to about 48 hours.

39. A pharmaceutical composition comprising a fusion protein or a chimeric molecule of claim 1.

40. A method for treating a metabolic disorder in a subject comprising administering to a subject in need of such a treatment a pharmaceutical composition of claim 39, wherein said metabolic disorder is selected from the group consisting of obesity, diabetes, a heart disease, and a liver disease.
